(19) United States
(12) Patent Application Publication
(10) Pub. No.: US 2010/0087871 A1
(43) Pub. Date: Apr. 8, 2010

(54) VERTEBRAL FIXATION PLATE ASSEMBLY

(76) Inventor: Walter X. Loyola, Plano, TX (US)

Correspondence Address: STORM LLP, BANK OF AMERICA PLAZA, 901 MAIN STREET, SUITE 7100, DALLAS, TX, 75202, US

(21) Appl. No.: 12/246,648

(22) Filed: Oct. 7, 2008

(51) Int. Cl. A61B 17/70; A61B 17/04

(52) U.S. Cl. 606/280; 606/70; 606/286; 606/301

(57) ABSTRACT

A vertebral plate assembly is configured to be attached to an existing plate assembly for fixation of at least a first vertebrate to a second vertebrate in a spinal column. The existing plate assembly fixates the second vertebrate to one or more other vertebrae in the spinal column. The vertebral plate assembly includes a vertebral plate having a length along a central axis extending between a first end and a second end. The second end is sized to sit on at least a portion of one end of the existing plate assembly.

### FIELD OF THE INVENTION

The invention generally relates to instrumentation for spinal fixation. More specifically, the invention relates to a vertebral fixation plate assembly.

### BACKGROUND OF THE INVENTION

The spinal column consists of thirty three irregular shaped bones known as the vertebral column. FIG. 1 depicts a vertebral column 100 having an anterior side 104 and a posterior side 108.

A top view of a vertebra 200 is depicted in FIG. 2. The vertebra 200 has a main body region 204 and a spinous process region 208. An opening 212 between the main body region 204 and the spinous process region 208 allows a spinal cord to pass.

FIG. 3A shows the main body regions 304 and 308 separated by an intervertebral disk 312. The intervertebral disk 312 resides in the intervertebral space 314. The main body region 304 (or 308) is drum-shaped having a cylindrical side section 306 and flat base sections 316 and 320.

FIG. 3B shows a perspective view of the main body region 304 with the cylindrical side section 306 and flat base sections 316 and 320. The intervertebral disk 312 is attached to the flat base sections of the main body regions. The intervertebral disk 312 cushions and softens the forces created by walking or jumping that might otherwise fracture the vertebrae.

### SUMMARY OF THE INVENTION

A vertebral plate assembly is configured to be attached to an existing plate assembly for fixation of at least a first vertebrate to a second vertebrate in a spinal column. The existing plate assembly fixates the second vertebrate to one or more other vertebrae in the spinal column. The vertebral plate assembly includes a vertebral plate having a length along a central axis extending between a first end and a second end. The second end is sized to sit on at least a portion of one end of the existing plate assembly.

The vertebral plate assembly includes a pair of first openings proximate to the first end. Each first opening defines a circular hole on a respective side of the central axis. The vertebral plate assembly includes a pair of second openings proximate to the second end. Each second opening defines a circular hole on a respective side of the central axis. Each second opening is aligned to a respective opening on the existing plate assembly when the second end sits on at least the portion of one end of the existing plate assembly.

The vertebral plate assembly includes a central opening defining a hole along the central axis between the first and second openings. A pair of first bone engaging fasteners each extends through one of the respective first opening of the vertebral plate to engage the first vertebrate and to attach the first vertebrate to the vertebral plate. A pair of second bone engaging fasteners each extends through one of the second opening of the vertebral plate and through one of the opening of the existing plate assembly to engage the second vertebrate and to attach the second vertebrate to the vertebral plate and the existing plate assembly.

The vertebral plate assembly includes a first locking hole along the central axis between the first pair of openings. The first locking hole is sized to house a first locking tab to hold the first pair of bone engaging fasteners in a locked position. The vertebral plate assembly includes a second locking hole along the central axis between the second pair of openings. The second locking hole is sized to house a second locking tab to hold the second pair of bone engaging fasteners in a locked position.

### BRIEF DESCRIPTION OF THE DRAWINGS

For a more complete understanding of the features and advantages of the present invention, reference is now made to the detailed description of the invention along with the accompanying figures and in which:

FIG. 1 depicts a vertebral column.

FIG. 2 depicts a top view of a vertebra.

FIG. 3A shows main body regions of a vertebra separated by an intervertebral disk.

FIG. 3B shows a perspective view of a main body region with the flat base sections.

FIG. 4 illustrates a vertebral plate assembly in accordance with one embodiment.

FIG. 5 illustrates the vertebral plate assembly being attached to an existing plate.

FIG. 6 shows a cross-sectional view of the vertebral plate.

### DETAILED DESCRIPTION OF THE EMBODIMENTS

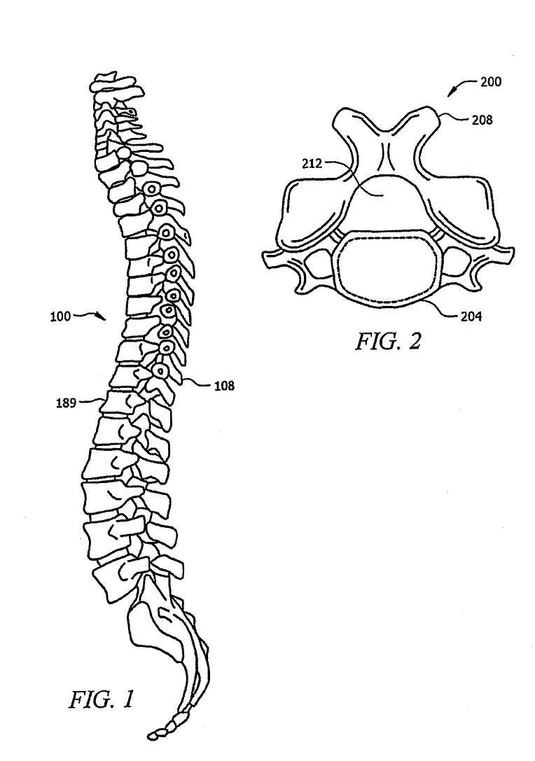

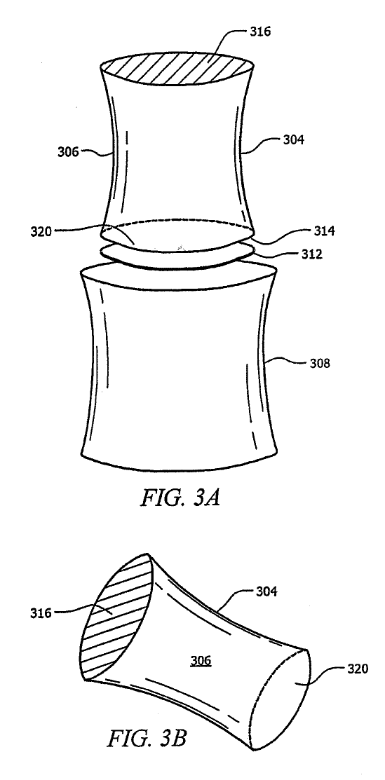

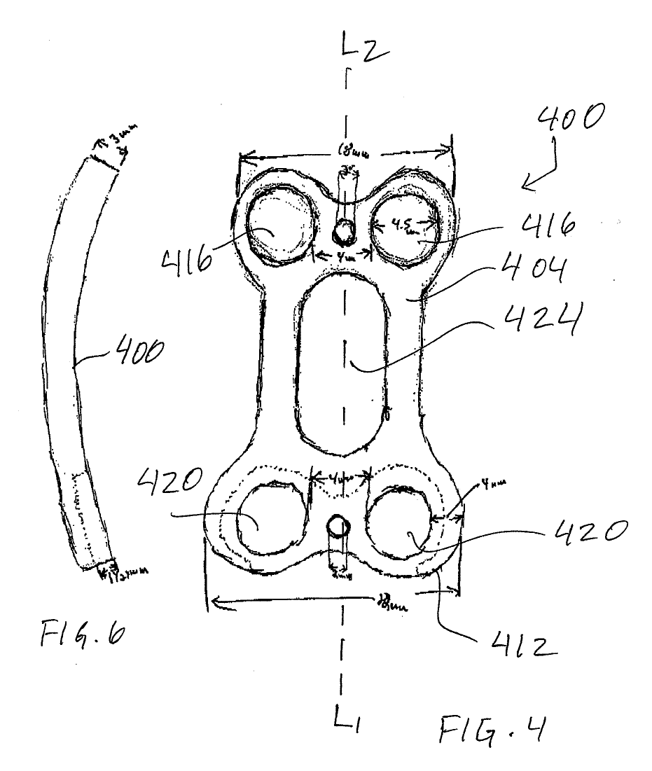

FIG. 4 illustrates a vertebral plate assembly 400 in accordance with one embodiment. The vertebral plate assembly 400 is configured to be attached to an existing plate assembly (not shown in FIG. 1) for fixation of at least a first vertebrate to a second vertebrate in a spinal column. The vertebral plate assembly 400 includes a vertebral plate 404 having a length along a central axis L1-L2 extending between a first end 408 and a second end 412. The second end 412 is sized to sit on at least a portion of one end of an existing plate assembly (not shown in FIG. 4).

The vertebral plate assembly 400 includes a pair of first openings 416 proximate to the first end 408. Each first opening 416 defines a circular hole on a respective side of the central axis L1-L2. The vertebral plate assembly 400 includes a pair of second openings 420 proximate to the second end 412. Each second opening 420 defines a circular hole on a respective side of the central axis L1-L2. Each second opening 420 is aligned to a respective opening on the existing plate assembly (not shown in FIG. 4) when the second end 412 sits on at least the portion of one end of the existing plate assembly. A central opening 424 defines a hole along the central axis between the first and second openings. The vertebral plate assembly 400 may be made from implantable steel, titanium or any other suitable implantable material.

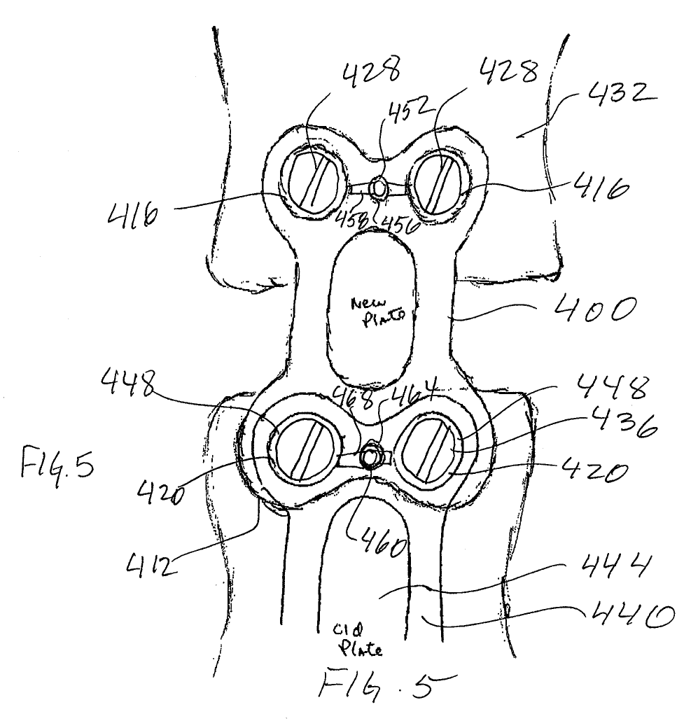

As shown in FIG. 5, a pair of first bone engaging fasteners 428 each extends through one of the respective first opening 416 of the vertebral plate 400 to engage a first vertebrate 432. The first bone engaging fasteners 428 attach the first vertebrate 432 to the vertebral plate 400.

A pair of second bone engaging fasteners 436 each extends through one of the second opening 420 of the vertebral plate 400 and through one of the opening of an existing plate assembly 440 to engage a second vertebrate 444. As shown in FIG. 5, each second opening 420 is aligned to a respective opening 448 on the existing plate assembly 440 when the second end 412 sits on the portion of one end of the existing plate assembly 440. Thus, the second bone engaging fasteners 436 attach both the vertebral plate 400 and the existing plate assembly 440 to the second vertebrate 444. The second end 412 of the vertebral plate 400 is sized to fit over the portion of the existing plate assembly 440, with the openings aligned to thereby allow the second bone engaging fasteners 436 to extend through the openings to engage the second vertebrate 444.

In one embodiment, a first locking hole 452 is formed along the central axis between the first pair of openings 416. The first locking hole 452 is sized to house a first locking tab 456 to retain the first pair of bone engaging fasteners 428 in a locked position. In one embodiment, the locking tab 456 has an unlocked and a locked position. In the unlocked position, the locking tab 456 allows the first bone engaging fasteners 428 to be removed. For example, if the first bone engaging fasteners 428 are threaded, the locking tab 456 may be adjusted to the unlocked position to unscrew and remove the first bone engaging fasteners 428 from the first pair of openings 416.

After the first bone engaging fasteners 428 are inserted into the first pair of openings 416, the locking tab 456 is adjusted to the locked position to prevent the first bone engaging fasteners 428 from loosening. In one embodiment, the locking screw 456 includes a blade 458 that engages the first bone engaging fasteners 428 to prevent from becoming loose. It will be appreciated that due to the movement of the vertebrae over a period of time, the bone engaging fasteners 428 may become unfastened or loose, causing the first vertebrate 432 to become detached from the second vertebrate 444. The locking tab 456 prevents the first bone engaging fasteners 416 from becoming unfastened.

A second locking hole 460 is formed along the central axis between the second pair of openings 420. The second locking hole 460 is sized to house a second locking tab 464 to retain the second pair of bone engaging fasteners 436 in a locked position. In one embodiment, the second locking tab 464 has an unlocked and a locked position. In the unlocked position, the second locking tab 464 allows the second bone engaging fasteners 436 to be removed. For example, if the second bone engaging fasteners 436 are threaded, then the second locking tab 464 may be adjusted to the unlocked position to unscrew and remove the second bone engaging fasteners 436 from the second pair of openings 420.

After the second bone engaging fasteners 436 are inserted into the second pair of openings 420, the second locking tab 464 is adjusted to the locked position to prevent the second bone engaging fasteners 436 from loosening or becoming unfastened. In one embodiment, the second locking tab 464 includes a blade 468 that engages the second bone engaging fasteners 436 to prevent from becoming loose or unfastened.

FIG. 6 shows a cross-sectional view of the vertebral plate 400 along its length. As will be appreciated, the vertebral plate 400 is curved along its length to accommodate for the curvature of the human spinal column or vertebrae. The vertebral plate 400 may have a uniform thickness along its length or the thickness along the length may vary. For example, thickness at the second end may be different than the thickness at the first end of the vertebral plate 400 in order to fit with the existing plate 440.

It will be appreciated that the vertebral plate assembly 400 enables the fixation of the first vertebra 432 to the second vertebra 444 that would otherwise be difficult because of the presence of the existing plate 440. As discussed before, the existing plate 440 fixates the second vertebra 444 to one or more other vertebrae in the spinal column. Subsequent to the implantation of the existing plate 440, if it becomes necessary to fixate the first vertebra 432 to the second vertebra 444, the vertebral plate assembly 400 enables the fixation of the first vertebra 432 to the second vertebra 444 without the removal of the existing plate 440. The vertebral plate assembly 400 is designed to attach to the existing plate 440 to fixate the first vertebra 432 to the second vertebra 444. In accordance with one method, bone engaging fasteners from the existing plate 440 are removed and the vertebral plate 400 is set partially over the existing plate 440 so that the second openings 420 of the vertebral plate 400 are aligned with the openings of the existing plate 440. Next, bone engaging fasteners 436 are inserted through the first openings of the vertebral plate 400 and through the openings of the existing plate 440 to engage the second vertebra 444.

It will be understood by those skilled in the art that the shape and dimension of the vertebral plate assembly 400 will vary depending on the shape and dimension of the existing plate 440. Various existing plates will require different vertebral plate assembly 400.

While the compositions, structures, apparatus and methods of this invention have been described in terms of preferred embodiments, it will be apparent to those of skill in the art that variations may be applied to the compositions, structures, apparatus and/or methods and in the steps or in the sequence of steps of the method described herein without departing from the concept, spirit and scope of the invention. All such substitutes and modifications apparent to those skilled in the art are deemed to be within the spirit, scope and concept of the invention as defined by the appended claims.

What is claimed is:

1. A vertebral plate assembly configured to be attached to an existing plate assembly for fixation of at least a first vertebrate to a second vertebrate in a spinal column, the existing plate assembly fixating the second vertebrate to one or more other vertebrae in the spinal column, the vertebral plate assembly comprising:
a vertebral plate having a length along a central axis extending between a first end and a second end, the second end being sized to sit on at least a portion of one end of the existing plate assembly;
a pair of first openings proximate to the first end, each first opening defining a circular hole on a respective side of the central axis;
a pair of second openings proximate to the second end, each second opening defining a circular hole on a respective side of the central axis, each second opening being aligned to a respective opening on the existing plate assembly when the second end sits on at least the portion of one end of the existing plate assembly;
a central opening defining a hole along the central axis between the first and second openings;
a pair of first bone engaging fasteners each extending through one of the respective first opening of the vertebral plate to engage the first vertebrate and to attach the first vertebrate to the vertebral plate;
a pair of second bone engaging fasteners each extending through one of the second opening of the vertebral plate and through one of the opening of the existing plate assembly to engage the second vertebrate and to attach the second vertebrate to the vertebral plate and the existing plate assembly;
a first locking hole along the central axis between the first pair of openings, the first locking hole sized to house a first locking tab to hold the first pair of bone engaging fasteners in a locked position;
a second locking hole along the central axis between the second pair of openings, the second locking hole sized to house a second locking tab to hold the second pair of bone engaging fasteners in a locked position.

2. The vertebral plate assembly of claim 1, wherein the first openings are threaded to receive the first bone engaging fasteners having bone engaging threads thereon.

3. The vertebral plate assembly of claim 1, wherein the second openings are threaded to receive the second bone engaging fasteners having bone engaging threads thereon.

4. The vertebral plate assembly of claim 1, wherein the first and second locking holes are circular.

5. The vertebral plate assembly of claim 1, wherein the diameter of each of the second opening is equal to the diameter of the respective opening of the existing plate assembly.

6. The vertebral plate assembly of claim 1, wherein the central axis extending between a first end and a second end of the vertebral plate is aligned with a central axis of the existing plate assembly.

7. The vertebral plate assembly of claim 1, wherein the vertebral plate assembly spans a spinal disk between the first and second vertebrae.

8. A bone fixation system for attaching a first vertebrate to a second vertebrate in a spinal column, the second vertebrate being fixated to one or more other vertebrae in the spinal column by an existing plate, the bone fixation system comprising:
a vertebral plate configured to be attached to the existing plate to fixate the first vertebrate to the second vertebrate, the vertebrate plate having a length along a central axis extending between a first end and a second end, the second end being sized to sit on at least a portion of one end of the existing plate;
a pair of first openings proximate to the first end, each first opening defining a circular hole on a respective side of the central axis;
a pair of second openings proximate to the second end, each second opening defining a circular hole on a respective side of the central axis, each second opening being aligned to a respective opening on the existing plate when the second end sits on at least the portion of one end of the existing plate;
a central opening defining a hole along the central axis between the first and second openings;
a pair of first bone engaging fasteners each extending through one of the respective first opening of the vertebral plate to engage the first vertebrate and to attach the first vertebrate to the vertebral plate;
a pair of second bone engaging fasteners each extending through one of the second opening of the vertebral plate and through one of the opening of the existing plate to engage the second vertebrate and to attach the second vertebrate to the vertebral plate and the existing plate;
a first locking hole along the central axis between the first pair of openings, the first locking hole sized to house a first locking tab to hold the first pair of bone engaging fasteners in a locked position;
a second locking hole along the central axis between the second pair of openings, the second locking hole sized to house a second locking tab to hold the second pair of bone engaging fasteners in a locked position.

9. The bone fixation system of claim 8, wherein the first and second locking holes are circular.

10. The bone fixation system of claim 8, wherein the diameter of each of the second opening is equal to the diameter of the respective opening of the existing plate.

11. The bone fixation system of claim 8, wherein the central axis extending between a first end and a second end of the vertebral plate is aligned with a central axis of the existing plate.
